(19) United States
(12) Patent Application Publication
(10) Pub. No.: US 2013/0102662 A1
(43) Pub. Date: Apr. 25, 2013

(54) USE OF INDUCIBLE NITRIC OXIDE SYNTHASE INHIBITORS AND NITRIC OXIDE SCAVENGERS TO INHIBIT POST-TRAUMATIC IMMUNODEPRESSION

(71) Applicant: University of Pittsburgh - of the Commonwealth System of Higher Education, Pittsburgh, PA (US)

(72) Inventors: Sophie Darwiche, Pittsburgh, PA (US); Timothy R. Billiar, Presto, PA (US)

(73) Assignee: University of Pittsburgh - of the Commonwealth System of Higher Education, Pittsburgh, PA (US)

(21) Appl. No.: 13/707,066

(22) Filed: Dec. 6, 2012

Related U.S. Application Data
(63) Continuation of application No. PCT/US2011/039778, filed on Jun. 9, 2011.

(51) Int. Cl. A61K 31/185; A61K 31/155

(52) U.S. Cl. CPC A61K 31/185; A61K 31/155
USPC 514/476; 514/637

(57) ABSTRACT

The present invention relates to methods and compositions that inhibit the effects of nitric oxide for inhibiting post-traumatic immunodepression. It is based, at least in part, on the discovery that, in a murine model system of trauma, genetic or chemical reduction of nitric oxide inhibited the development of immunodepression. Accordingly, the present invention provides for methods and compositions to be administered to subjects, post-trauma, that inhibit the effects of nitric oxide by inhibiting inducible nitric oxide synthase (iNOS) and/or scavenge nitric oxide.

### PRIORITY CLAIM

This application is a continuation of International Patent Application Serial No. PCT/US2011/039778, filed Jun. 9, 2011, and claims priority to U.S. Provisional Application Ser. No. 61/354,103, filed Jun. 11, 2010, both of which are hereby incorporated by reference in their entireties.

### GRANT INFORMATION

This invention was made with government support under grant number GM053789 awarded by the National Institutes of Health. The government has certain rights in the invention.

### 1. INTRODUCTION

The present invention relates to methods and compositions that inhibit the effects and/or production of nitric oxide which may be used for inhibiting post-traumatic immunodepression.

### 2. BACKGROUND OF THE INVENTION

After surgical or accidental trauma, a subject first exhibits a strong inflammatory response, but this is then followed by a dramatic depression of cell-mediated immunity (Angele and Chaudry, 2005, Langenbecks Arch Surg. 390:333-341; Menger and Vollmar, 2004, Arch Surgery 389(6):475-484). This trauma-induced immunodepression renders the subject vulnerable to localized infection as well as generalized sepsis and shock.

### 3. SUMMARY OF THE INVENTION

The present invention relates to methods and compositions that inhibit the effects and/or production of nitric oxide for inhibiting post-traumatic immunodepression. It is based, at least in part, on the discovery that, in a murine model system of trauma, genetic or chemical reduction of nitric oxide inhibited the development of immunodepression. Accordingly, the present invention provides for methods and compositions to be administered to subjects, pre- or post-trauma, that inhibit the effects of nitric oxide by inhibiting inducible nitric oxide synthase (“iNOS”), which produces nitric oxide (“NO”), and/or scavenge NO, a free radical associated with multiple physiologic effects. In additional non-limiting embodiments, the present invention relates to methods and compositions that inhibit the effects and/or production of nitric oxide for inhibiting post-sepsis immunodepression.

### 4. BRIEF DESCRIPTION OF THE FIGURES

FIG. 1. Splenocyte proliferation as measured by tritiated thymidine (3H-dT) in WT and iNOS knockout mice. All data: Mean±SEM, p<0.05*vs. respective unmanipulated controls (Student t-test).

FIG. 2A-B. (A) IFNγ release by splenocytes from WT and iNOS knockout mice.

(B) IL-2 release by splenocytes from WT and iNOS knockout mice. All data: Mean±SEM, p<0.05*vs. respective unmanipulated controls (Student t-test) of wild type mice, p<0.05 #vs. respective unmanipulated controls (Student T-test) for the iNOS −/− strain.

FIG. 3. Splenocyte proliferative responses of C57BL/6 mice with administration of 1400 W in response to stimulation to concanavalin A. All data: Mean±SEM, p<0.05*vs. respective unmanipulated controls (Student t-test).

FIG. 4. IFNγ (“IFN-g”) release by splenocytes from C57BL/6 mice following trauma with administration of 1400 W in response to stimulation by concanavalin A. All data: Mean±SEM, p<0.05*vs. respective unmanipulated controls (Student t-test).

FIG. 5. Splenocyte proliferation responses to stimulation by antiCD3e. 1400 W restores the proliferation response following trauma. All data: Mean±SEM, p<0.05*vs, respective unmanipulated controls (Student t-test).

FIG. 6. Splenocyte IFNγ (“IFN-g”) release in response to antiCD3e stimulation. All data: Mean±SEM, p<0.05*vs. respective unmanipulated controls (Student t-test).

FIG. 7. Results of RT-PCR studies measuring iNOS mRNA in control subjects and 1, 3, 6, 12 or 24 hours post-pseudofracture.

FIG. 8A-B. Immunofluoresence detection of iNOS in the spleen. iNOS:green stain, Nuclei:blue stain. (A) control baseline (B) 6 hours after pseudofracture trauma (arrowheads point to certain exemplary green stained areas).

FIG. 9A-B. (A) Splenocyte proliferation in C57B1/6 mice, measured as counts per minute (c.p.m.) of tritated thymidine (3H-dT) uptake after concanavalin A stimulation. All data: Mean±SEM, *=p<0.05 vs. unmanipulated controls; #=p<0.05 vs. laparotomy [pumps Veh]; ̂=p=0.05 vs. PF+laparotomy [pumps Veh]. (B) Splenocyte proliferation in C57B1/6 mice, measured as counts per minute (c.p.m.) of tritated thymidine (3H-dT) uptake after AntiCD3e stimulation. All data: Mean±SEM, *=p<0.05 vs. unmanipulated controls; #=p<0.05 vs. laparotomy [pumps Veh]; ̂=p=0.05 vs. PF+laparotomy [pumps Veh]. (B) Splenocyte proliferation in C57B1/6 mice, measured as counts per minute (c.p.m.) of tritated thymidine (3H-dT) uptake after AntiCD3e stimulation. All data: Mean±SEM, *=p<0.05 vs. unmanipulated controls; #=p<0.05 vs. laparotomy [pumps Veh]; ̂=p=0.05 vs. PF+laparotomy [pumps Veh].

FIG. 100A-B. (A) Splenocyte interferon-gamma release in C57B1/6 mice after concanavalin A stimulation. All data: Mean±SEM, *=p<0.05 vs. unmanipulated controls; #=p<0.05 vs laparotomy [pumps Veh], ̂=p<0.05 vs PF+laparotomy [pumps Veh]. (B) Splenocyte interferon-gamma release in C57B1/6 mice after AntiCD3e stimulation. All data: Mean+SEM, *=p<0.05 vs. unmanipulated controls; #=p<0.05 vs laparotomy [pumps Veh], ̂=p<0.05 vs PF+laparotomy [pumps Veh].

### 5. DETAILED DESCRIPTION OF THE INVENTION

For clarity of description, and not by way of limitation, the detailed description of the invention is divided into the following subsections:

(i) NO scavengers;

(ii) iNOS inhibitors; and

(iii) methods of treatment.

### 5.1 No Scavengers

A NO scavenger, according to the invention, is an agent that removes, deactivates or destroys NO.

Non-limiting examples of NO scavengers that are known in the art and which may be used according to the invention include dithiocarbamate compounds, including but not limited to NOX-100 (also known as Norathiol™, Medinox Inc., Carlsbad, Calif.), which are optionally complexed to a di-valent or trivalent transition metal ion such as iron, for example, N-methyl-D-glucamine dithiocarbamate (“MGD”) complexed with iron as (MGD)₂/Fe, and including those compounds set forth in U.S. Pat. Nos. 5,847,004; 5,741,815; 5,747,532; 5,756,540; 6,265,420 and 6,596,733. NOX-100 has the following chemical structure:

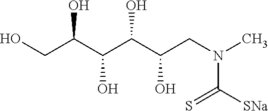

Other non-limiting examples of NO scavengers include 2-(4-carboxyphenyl)-4,4,5,5-tetramethylimidazoline-1-oxy]-3-oxide (“carboxy-PTIO”), as described in Cao et al., 2022, Eur. J. Pharmacol. 448:27-30; 2-phenyl-4,4,5,5-tetramethylimidazoline-1-oxy]-3-oxide (“PTIO”), as described in Maeda et al., 1994, J. Leuk. Biol. 56(5):588-592; cobinamide, as described in Broderick et al., 2005, J. Biol. Chem. 280:8678-8685; a curcuminoid compound such as curcumin, as described in Sreejayan, 1997, J. Pharm. Pharmacol. 49(1):105-107; diethyldithiocarbamate; proline dithiocarbamate; N-acetyl-L-cysteine; 5,5-dimethyl-1-pyrroline N-oxide; or AMD6221 (ruthenium [hydrogen (diethylenetrinitrilo)pentaacetato]chloride) as described in Hutchings et al., 2005, Eur. J. Pharmacol. 528:132-136.

The present invention provides for a composition comprising an effective amount of a NO scavenger to be used to inhibit the development of post-trauma immunodepression in a subject; such composition may further comprise a suitable pharmaceutical carrier and/or an agent with clinically beneficial activity, such as, but not limited to, a cytokine, an antibiotic, an analgesic, an iNOS inhibitor or a combination thereof.

### 5.2 iNOS Inhibitors

An iNOS inhibitor, according to the invention, is an agent that decreases or inhibits the activity of iNOS to produce NO. The iNOS gene is described, for example, in International Patent Application No. PCT/US95/07849 (Publication No. WO 96/00006).

Non-limiting examples of iNOS inhibitors that are known in the art and which may be used according to the invention, include:

1400 W, the chemical name of which is N-(3-(Aminomethyl)benzyl)acetamidine;

(N-[(1,3-benzodioxol-5-yl)methyl)-1-[2-(1H-imidazol-1-yl)pyrimidin-4-yl]-4-(methyoxycarbonyl)-piperazine-2-acetamide (Compound 2) and other compounds (e.g. Compounds 1 and 3) described in McMillan et al., 2000, Proc. Natl. Acad. Sci. U.S.A. 97214):1506-1511;

AMT hydrochloride, the chemical name of which is (4H-1,3-Thiazin-2-amine, 5,6-dihydro-6-methyl-, [monohydrochloride]. as described in Alexander et al., 2002, Hypertension 39:586-590;

2-aminopyridines, as described in Connolly et al., 2004, J. Med. Chem. 47(12):3320-3323; and

arginine derivatives such as L-NMMA, as described in U.S. Pat. Nos. 5,028,627 and 5,585,402.

Additional iNOS inhibitors may be identified by known assays, for example the following assay described in McMillan et al., 2000, Proc. Natl. Acad. Sci. U.S.A. 97(4): 1506-1511. A-172 human glioblastoma cells may be cultured in DMEM supplemented with 10 percent fetal bovine serum. The cells may then be plated into 96-well tissue culture plates at 100,000 cells per well. After culturing the cells for 24 hours, iNOS may be induced by the addition of 400 units/ml human IFNγ, 4 ng/ml of human IL-1β, and 40 ng/ml of human tumor necrosis factor α. Putative iNOS inhibitor may be added at the time of cytokine induction. NO production may be measured 22 hours after induction by mixing 0.125 ml of culture medium with an equal volume of Griess reagent as described in Stuehr et al., 1985, Proc. Natl. Acad. Sci. U.S.A. 82:7738-7742.

The present invention provides for a composition comprising an effective amount of an iNOS inhibitor to be used to inhibit the development of post-trauma immunodepression in a subject; such composition may further comprise a suitable pharmaceutical carrier and/or an agent with clinically beneficial activity, such as, but not limited to, a cytokine, an antibiotic, an analgesic, a NO scavenger or a combination thereof.

### 5.3 Methods of Treatment

The present invention provides a method of inhibiting post-trauma immunodepression in a subject who has suffered a traumatic injury, comprising administering, to the subject, an effective amount of an agent that inhibits the effects of nitric oxide (for example by inhibiting its production or by acting as a NO scavenger). Post-trauma immunodepression is a condition whereby there is a decrease in cell-mediated immunity, for example a decrease in the type 1 T-helper cell response and a reduction in (IL)-2, interferon (IFN)-gamma, and IL-12 levels.

Accordingly, in non-limiting embodiments the invention provides a method for inhibiting a decrease in cell-mediated immunity in a subject who has suffered a traumatic injury, comprising administering, to the subject, an effective amount of an agent that inhibits the effects of nitric oxide.

In further non-limiting embodiments the invention provides a method for reducing the risk of infection in a subject who has suffered a traumatic injury, comprising administering, to the subject, an effective amount of an agent that inhibits the effects of nitric oxide. Infections for which the risk can be reduced include, but are not limited to, wound infection, pulmonary infection for example pneumonia, and sepsis.

In further non-limiting embodiments the invention provides a method for reducing the risk of sepsis in a subject who has suffered a traumatic injury, comprising administering, to the subject, an effective amount of an agent that inhibits the effects of nitric oxide.

As described below, the present invention may further be utilized in a subject who expects to undergo a traumatic injury, for example a surgery. As such, the present invention provides for a method of inhibiting post-trauma immunodepression in a subject who is expected to suffer a traumatic injury, comprising administering, to the subject, an effective amount of an agent that inhibits the effects of nitric oxide. In other embodiments the invention provides for a method for inhibiting a decrease in cell-mediated immunity in a subject who is expected to suffer a traumatic injury, comprising administering, to the subject, an effective amount of an agent that inhibits the effects of nitric oxide. In other embodiments the invention provides for a method for reducing the risk of infection in a subject who is expected to suffer a traumatic injury, comprising administering, to the subject, an effective amount of an agent that inhibits the effects of nitric oxide.

Trauma may be surgical or non-surgical (e.g., accidental). A trauma may comprise an incision, laceration, tear, burn, or crushing injury of a tissue, where the tissue may be skin, non-cardiac muscle, bone, and/or an internal organ such as but not limited to the liver, lung, spleen, heart, gastrointestinal tract, or brain.

In one set of non-limiting embodiments, the agent is an iNOS inhibitor. In another set of non-limiting embodiments, the agent is a NO scavenger. In another set of non-limiting embodiments, the agent is a combination of an iNOS inhibitor and a NO scavenger.

The subject may be a human or a non-human subject such as a companion animal (e.g., dog, cat, horse, etc.) farm animal or laboratory animal.

An effective amount of an iNOS inhibitor or an NO scavenger is that amount that inhibits trauma-induced immunodepression, as may be measured, for example, by the capacity of T lymphocytes to proliferate in response to a stimulus or by cytokine production in response to a stimulus (e.g., interferon gamma, interleukin 2, or interleukin 12 production). As a non-limiting example, by inhibiting immunodepression, an agent used according to the invention may result in an increased capacity of T lymphocytes to proliferate and/or produce cytokine (e.g., interferon gamma, interleukin 2, or interleukin 12) in response to antigenic stimulation relative to a control trauma patient who has not been treated with the agent. In certain non-limiting embodiments of the invention, an effective amount is that amount which achieves a reduction in NO or NO metabolites such as nitrate or nitrite of at least about 10 percent, or at least about 25 percent, or at least about 40 percent, or at least about 60 percent, in a suitable assay system. NO reducing dosages of certain NO scavengers and iNOS inhibitors listed herein are known in the art or may be determined by standard techniques. Non-limiting parameters that may be measured to show reduction of nitric oxide levels include, but are not limited to, plasma nitrate levels and plasma nitrite levels. See, for example, Roza et al., 2000, Transplantation 69:227-231.

The inhibitory agent may be administered by any route known in the art, including but not limited to subcutaneous, intradermal, intramuscular, intravenous, intraarterial, nasal, pulmonary, intrathecal, intraarticular, intraperitoneal, etc.

The inhibitory agent(s) may be comprised in a suitable pharmaceutical carrier, for example, but not limited to, normal saline or water, or may be comprised in a sustained release formulation.

In certain non-limiting embodiments, the dithiocarbamate NOX-100 may be administered as a bolus injection in single or multiple doses. For example, but not by way of limitation, where the subject is a human subject, the bolus dose of a dithiocarbamate such as NOX-100 may be between about 1 and 75 mg/kg, or between about 50 and 75 mg/kg, or between about 1 and 50 mg/kg, or between 1 and 20 mg/kg, or between 1 and 10 mg/kg, or between 1 and 5 mg/kg, or a human dose equivalent to 40 mg/kg given to a mouse. Those of skill in the art readily recognize that the selected dosage level will depend upon a variety of factors including the activity of the particular active agents employed, the route of administration, the time of administration, the rate of excretion of the particular compositions being employed, the duration of the treatment, other drugs, compounds and/or materials used in combination with the particular active agent employed, the age, sex, weight, condition, general health and prior medical history of the patient being treated, and like factors well known in the medical arts. In non-limiting embodiments, the number of bolus doses which may be administered in treating a trauma patient may be one, two, three, four, five, six, seven, eight, nine, ten doses, eleven, twelve, or more doses. In non-limiting embodiments, the interval between doses, where multiple doses are administered, may be the same or different between doses and may be between 0.5 and 1 hour (inclusive of endpoints for this and all the following examples), between 1 and 2 hours, between 2 and 3 hours, between 3 and 4 hours, between 4 and 6 hours, between 6 and 8 hours, between 8 and 10 hours, between 10 and 12 hours, or between 12 and 24 hours, or between 24 and 36 hours or between 36 and 48 hours.

In certain non-limiting embodiments, a dithiocarbamate such as NOX-100 may be administered as a continuous infusion. For example, but not by way of limitation, where the subject is a human, the infusion may deliver between about 1 and 50 mg/kg/hr, or between 1 and 20 mg/kg/hr, or between 1 and 10 mg/kg/hr, or between 1 and 5 mg/kg/hour, or a human dose equivalent to 30 mg/kg/hr to a mouse, of a dithiocarbamate such as NOX-100. In non-limiting embodiments, the continuous infusion may be administered for a period of between 0.5 and 1 hour (inclusive of endpoints for this and all the following examples), between 1 and 2 hours, between 2 and 3 hours, between 3 and 4 hours, between 4 and 6 hours, between 6 and 8 hours, between 8 and hours, between 10 and 12 hours, or between 12 and 24 hours, or between 24 and 36 hours or between 36 and 48 hours. The continuous infusion may optionally be interrupted by an infusion-free interval or may be repeated, according to the clinical response of the subject receiving the infusion.

In another non-limiting embodiment of the invention, 1400 W may be administered as a bolus injection in single or multiple doses. For example, but not by way of limitation, where the subject is a human subject, the bolus dose of 1400 W may be between about 0.01 and 5 mg/kg, or between 0.1 and 1 mg/kg, or a human dose equivalent to 5 mg/kg given to a mouse. In non-limiting embodiments, the number of bolus doses which may be administered in treating a trauma patient may be one, two, three, four, five, six, seven, eight, nine, ten doses, eleven, twelve, or more doses. In non-limiting embodiments, the interval between doses, where multiple doses are administered, may be the same or different between doses and may be between 0.5 and 1 hour (inclusive of endpoints for this and all the following examples), between 1 and 2 hours, between 2 and 3 hours, between 3 and 4 hours, between 4 and 6 hours, between 6 and 8 hours, between 8 and 10 hours, between 10 and 12 hours, or between 12 and 24 hours, or between 24 and 36 hours or between 36 and 48 hours.

In certain non-limiting embodiments, 1400 W may be administered as a continuous infusion. For example, but not by way of limitation, where the subject is a human, the infusion may deliver between about 0.01 and 5 mg/kg/hr, or between about 0.1 and 1 mg/kg/hr, of 1400 W. In non-limiting embodiments, the continuous infusion may be administered for a period of between 0.5 and 1 hour (inclusive of endpoints for this and all the following examples), between 1 and 2 hours, between 2 and 3 hours, between 3 and 4 hours, between 4 and 6 hours, between 6 and 8 hours, between 8 and 10 hours, between 10 and 12 hours, or between 12 and 24 hours, or between 24 and 36 hours or between 36 and 48 hours. The continuous infusion may optionally be interrupted by an infusion-free interval or may be repeated, according to the clinical response of the subject receiving the infusion

The iNOS inhibitor or NO scavenger may be administered prior to trauma (eg pre-operatively, concurrently with trauma (eg intra-operatively), or following trauma). For example, and not by way of limitation, the iNOS inhibitor and/or NO scavenger may be administered within 24 hours before surgery, or within 12 hours before surgery, or within 6 hours of surgery, or within 5 hours of surgery, or within 4 hours of surgery, or within 3 hours of surgery, or within 2 hours of surgery, or within 1 hour of surgery, or during the surgical procedure, or may be administered within 2 hours after surgery or other trauma, or within 6 hours after surgery or other trauma, or within 8 hours after surgery or other trauma, or within 10 hours after surgery or other trauma, or within 12 hours after surgery or other trauma, or within 24 hours after surgery or other trauma, or within 36 hours after surgery or other trauma.

The success of the method in inhibiting post-trauma immunodepression may be assessed using a marker of cell-mediated activity. For example, but not by way of limitation, the ability for lymphocytes of the subject, in response to stimulus, to produce a cytokine such as IFNγ relative to healthy control lymphocytes may be used to assess the degree of immunodepression, if any, in a post-trauma subject.

The methods and agents set forth above may, in further non-limiting embodiments of the invention, be used in inhibiting post-sepsis immunodepression in a subject who has developed sepsis, comprising administering, to the subject, an effective amount of an agent that inhibits the effects of nitric oxide. “Post-sepsis” means after the onset of sepsis and does not require that the sepsis has been successfully treated. The agents that may be used and the amounts effective in inhibiting the effects of nitric oxide are as set forth above.

### 6. EXAMPLE

### Inducible NOS Contributes to Late Post-Traumatic Immune Dysfunction

To study the delayed consequence to severe tissue trauma on immune function, a novel pseudofracture model was developed which recapitulates the systemic and end organ responses observed following bilateral femur fracture. Using the model the effect of iNOS on delayed immune dysfunction was evaluated.

### 6.1 Materials and Methods

Male wild type (WT) C57BL/6, iNOS knockout(−/−) mice, weighing 20-30 g were used in this study. The mice were divided into two groups (n=6-10), a pseudofracture trauma group and a control group. Wild type C57BL/6 mice also received 1400 W, [N-(3-(Aminomethyl)benzyl)acetamidine], a potent iNOS inhibitor, (5 mg/kg) intraperitonally according to two dosing strategies: single dose at time of trauma, or two doses, one at time of trauma and the second at 24 hrs following. The previously validated pseudofracture trauma model involves soft tissue injury in the form of a muscle crush injury to the thigh musculature bilaterally, followed by an injection of a crushed bone solution (prepared from a syngeneic age- and weight matched donor mouse). Control mice received no experimental manipulation.

The pseudofracture model was designed with the intent of recreating features of long bone fracture; injured muscle and soft tissue are exposed to damaged bone and bone marrow without breaking the native bone. This model allows the long term survival of the mice, without the complications of prolonged anesthesia and intricate fracture fixation techniques, hence permitting study of late term post-traumatic immune responses.

Mice were euthanized at 6 and 48 hours. Measurements at the early 6 hour time point included assessment of systemic inflammation and remote organ dysfunction. Whole blood was collected to evaluate plasma cytokine and liver enzyme levels. The late 48 hour time point included assessment of splenocyte responses; both splenocyte proliferation and Th1 cytokine release (interferon gamma (“IFNγ”), interleukin-2 (“IL-2”)) were measured in response to two T cell mitogens: concanavalin A (2.5 μg/ml) and antiCD3e (1 μg/ml), and total splenocyte cultures were used. Splenocyte proliferation was assessed using tritiated thymidine uptake as counts per minute (c.p.m.). Statistical significance (p<0.05) was assessed by Student's t-test and ANOVA.

### 6.2 RESULTS

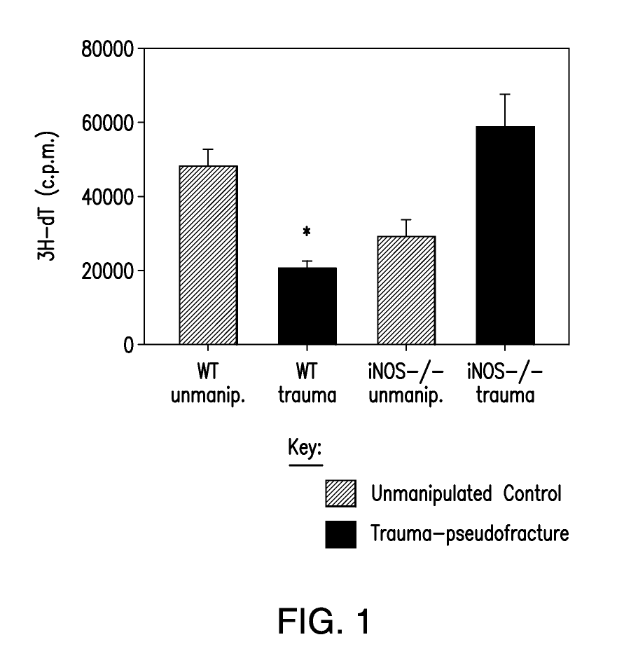

As shown in FIG. 1, a significant (p<0.05) decrease (23,886±1,880 c.p.m.) in splenocyte proliferation post-pseudofracture trauma was seen in the wild type mice at 48 hours in comparison to controls (49,229±5,996 c.p.m.). Splenocyte proliferation in iNOS knockout mice was similar (p<0.05) to wild type mice at baseline (29,497.0±4,939 c.p.m.). However, in contrast to wild type mice, splenocytes from iNOS knockout mice proliferated at the same rate as uninjured mice even after injury (59,154.9±8,695 c.p.m.).

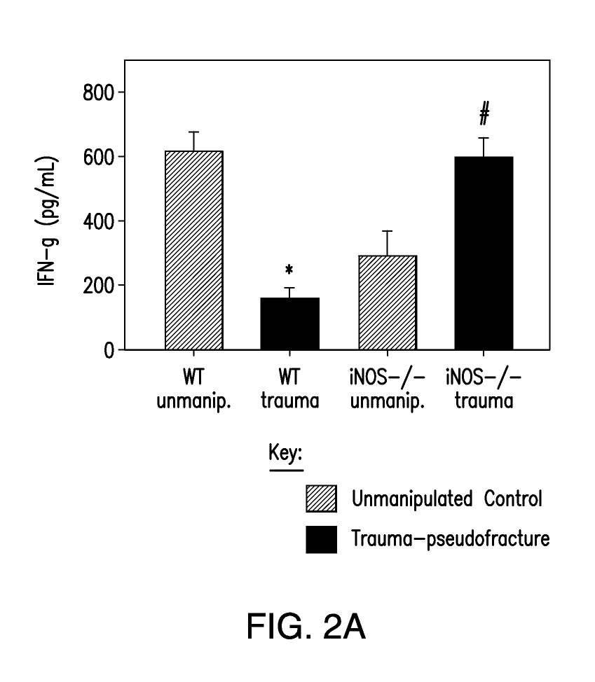

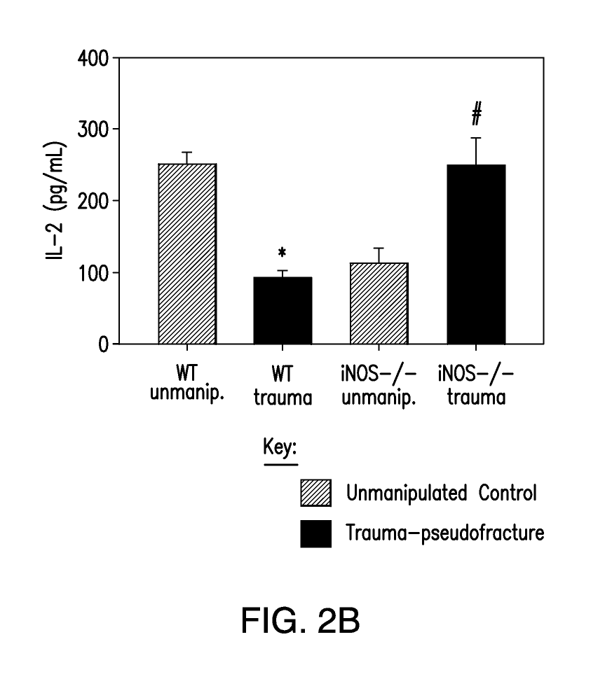

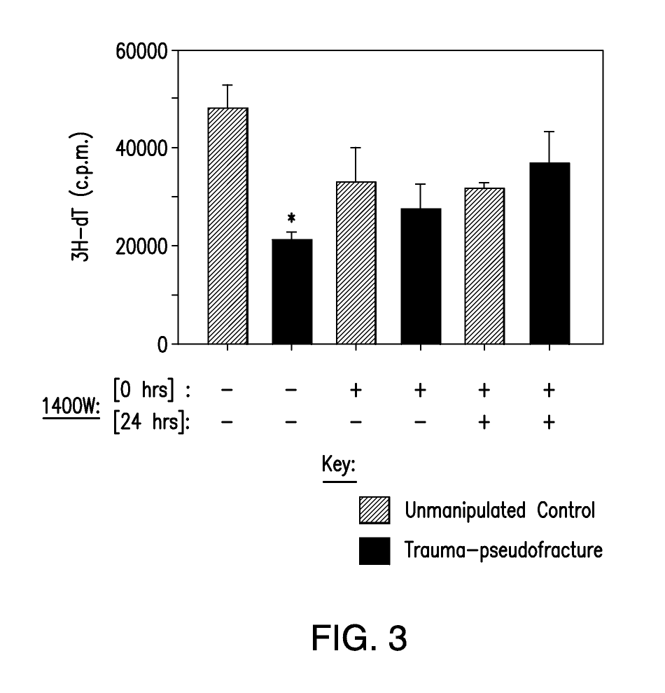

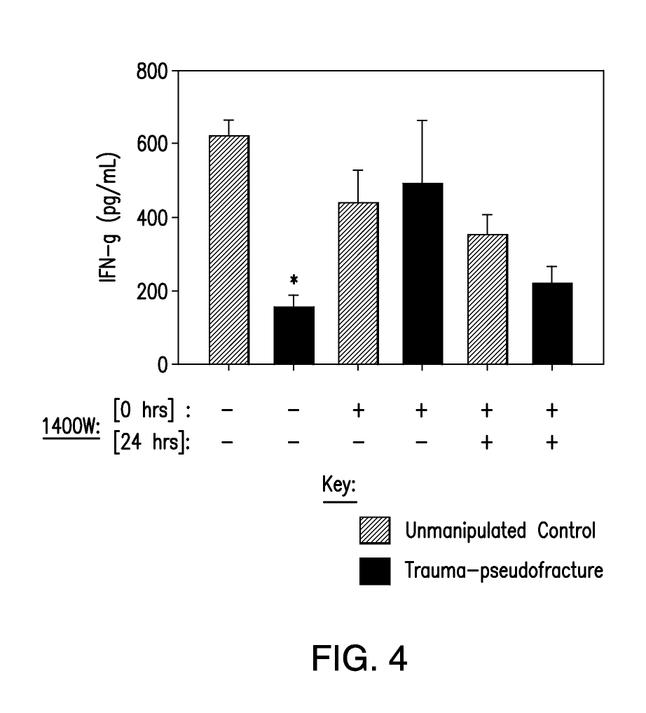

As shown in FIGS. 2A and 2B, splenocyte release of Th1 cytokines was significantly (p<0.05) decreased (IFNγ: 158.1±31.8 pg/ml; IL-2: 94.8±8.5 pg/ml) in injured wild type mice in comparison with controls as expected (IFN-g: 621.9±50.8 pg/ml; IL-2: 250.2±18.5 pg/ml), following concanavalin A stimulation. This suppression was not seen in splenocytes from injured iNOS deficient mice (IFN-g: 600.2±55.4 pg/ml; IL-2: 253.6±34.9 pg/ml) compared with iNOS deficient controls (IFN-g: 292.67±75.6 pg/ml; IL-2: 117.0±19.7 pg/ml). Concanavalin A stimulated splenocyte responses of mice that received 1400 W following trauma were not significantly reduced in comparison with baseline controls, confirming findings shown with iNOS knockout mice (FIG. 3). Concanavalin A stimulated splenocyte cytokine release (IFNγ) by C57BL/6 mice that received 1400 W was not significantly reduced following trauma as shown in comparison to the wild type mice that had not received treatment (FIG. 4).

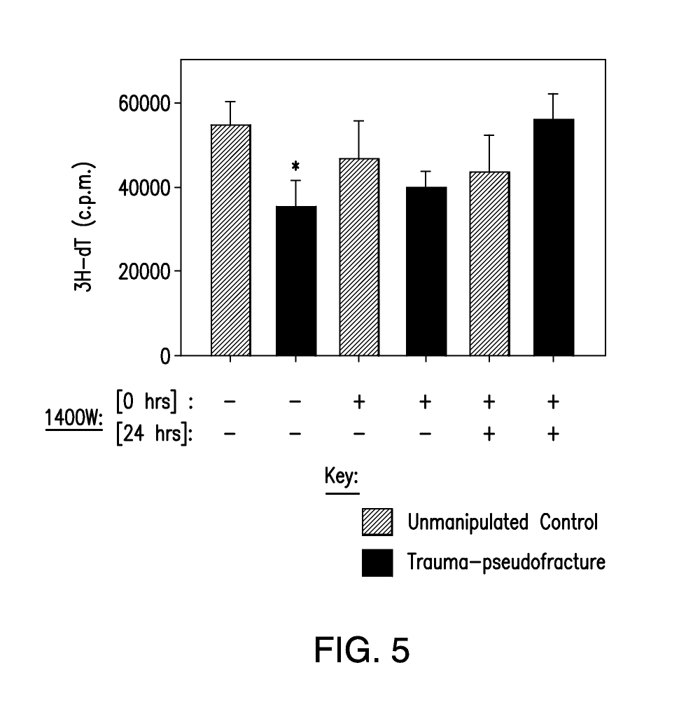

Stimulation of splenocytes with antiCD3e showed similar responses, though at slightly elevated values. FIG. 5 shows the splenocyte proliferative responses to antiCD3e by WT mice following trauma and with the administration of 1400 W. Significant depression in proliferation was seen, as expected, with the WT mice after trauma, but the proliferative responses were restored with the administration of 1400 W after trauma.

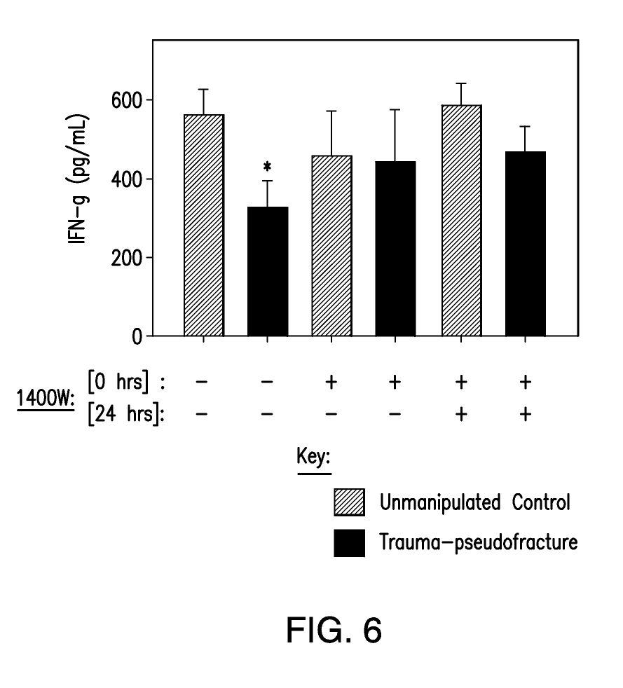

FIG. 6 shows the interferon-gamma release by splenocytes after antiCD3e stimulation. Again, administration of 1400 W to mice following trauma, restored the splenocyte function; no depression in cytokine release was found when 1400 W was administered.

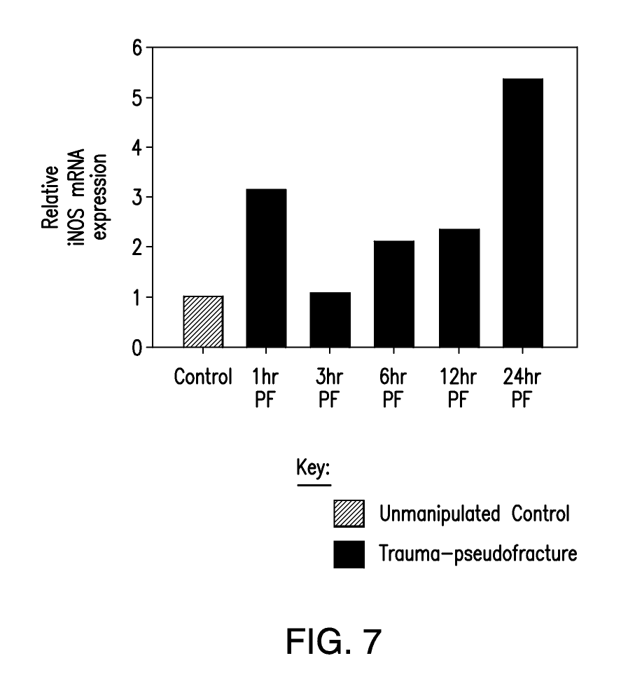

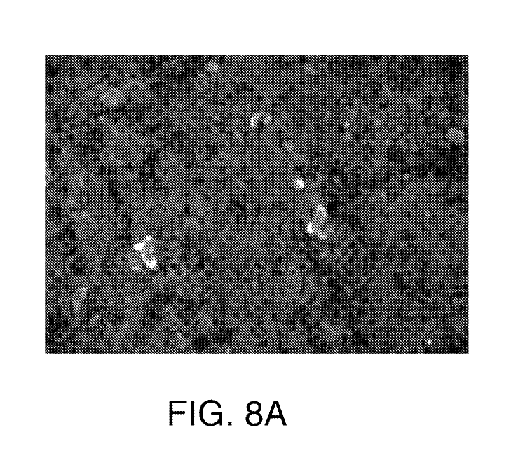

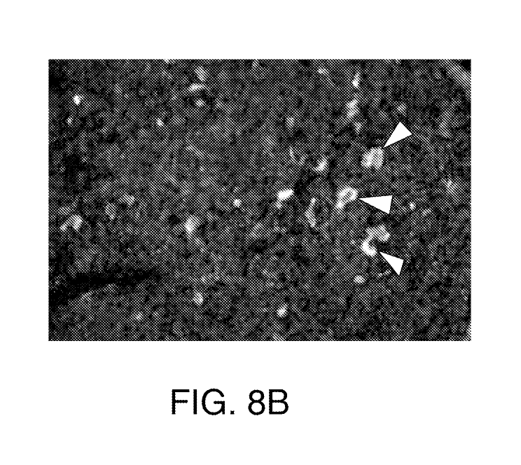

FIG. 7 shows the RT-PCR analysis of iNOS mRNA expression in the spleen over a time course out to 24 hours relative to controls. An early peak at one hour was followed by a second peak at the 24 hours timepoint. Immunofluoresence detection of iNOS in the spleen showed localisation to macrophages (FIG. 8A, 8B). A minimal level of iNOS expression was found at baseline, this increased at the 6 hour timepoint following trauma, but was not seen at 48 hours.

In conclusion, the failure of delayed immune dysfunction to develop in iNOS deficient animals indicates that iNOS contributes to the immune dysfunction seen in severe trauma. These genetic findings were corroborated by pharmacologic testing with 1400 W, a potent iNOS inhibitor, thereby confirming a role of iNOS signaling in late post-traumatic immune dysfunction. These findings reveal NO as a therapeutic target for modulation to limit injury-induced immune suppression.

### 7. EXAMPLE

### Use of Nitric Oxide Scavengers, Including the Compound NOX-100, to Inhibit And Prevent Post Injury Immunodepression

### 7.1 Materials and Methods

Male wild type (WT) C57BL/6 mice, weighing 20-30 g were used in this study. The mice were divided into six groups (n=5-10):

1. Unmanipulated controls

2. Pseudofracture (PF) 48 hrs

3. Laparotomy [pumps Veh]

4. Laparotomy [pumps NOX100]

5. PF+laparotomy [pumps Veh]

6. PF+laparotomy [pumps NOX100]

The injury model used was the previously validated pseudofracture (PF) trauma model that involves soft tissue injury in the form of a muscle crush injury to the thigh musculature bilaterally, followed by an injection of a crushed bone solution (prepared from a syngeneic age- and weight matched donor mouse). Mice then also received either NOX-100 at a dose of 30 mg/kg/hr or vehicle (Veh) over the 48 hour time period. A controlled, continuous drug delivery rate was established through the use of osmotic pumps inserted intraperitoneally by laparotomy. Laparotomy with pump insertion was performed at the time of trauma (0 hours). Please note that the laparotomy procedure itself is considered a significant trauma. Mice in the ‘Unmanipulated controls’ group received no experimental manipulation.

Mice were euthanized at a 48 hour time point. Measurements at this late time point included assessment of splenocyte responses. Splenocyte proliferation, a well established parameter of immune dysfunction was assessed. Measurements of splenocyte proliferative capacity were in response to two T cell mitogens: concanavalin A (2.5 μg/ml) and antiCD3e (1 μg/ml), and total splenocyte cultures were used. Splenocyte proliferation was assessed using tritiated thymidine (³H-dT) uptake as counts per minute (c.p.m.). Statistical significance (p<0.05) was assessed by Student's t-test and ANOVA.

### 7.2 Results

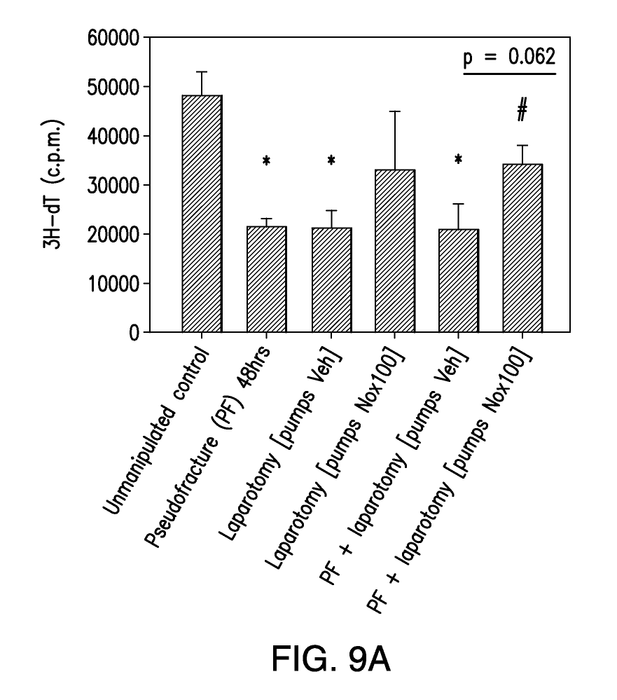

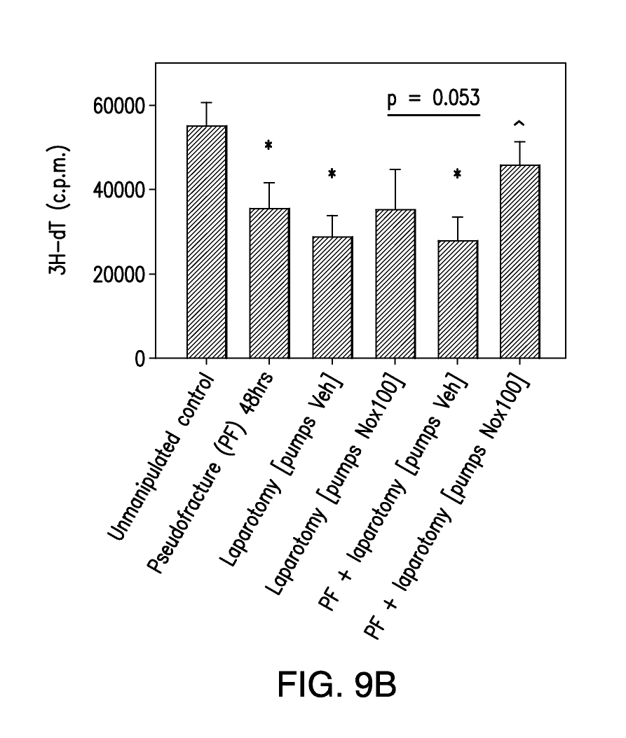

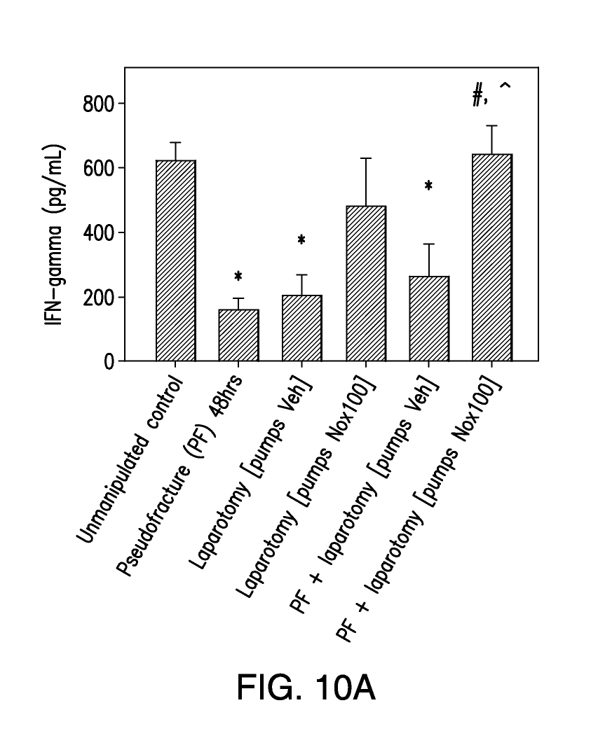

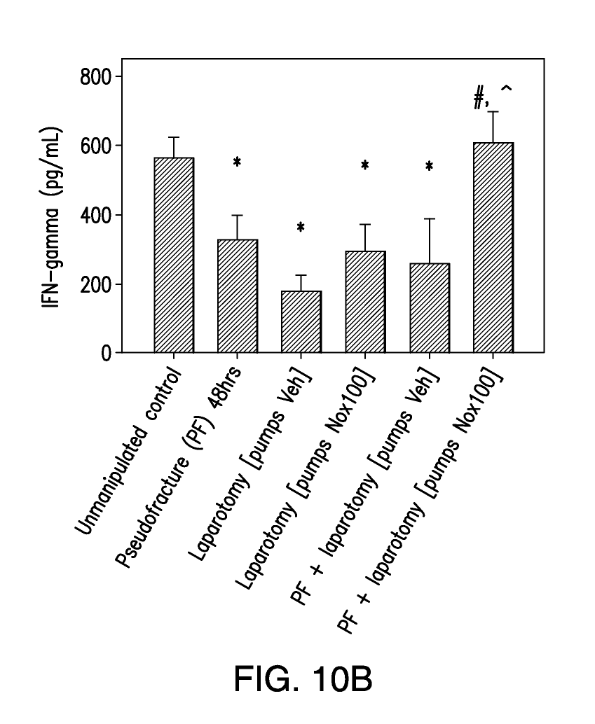

A significant decrease in splenocyte proliferation was seen at 48 hrs post injury in comparison to uninjured controls. FIG. 9A shows this significant (p<0.05) decrease (21258.9±1723.3 c.p.m.) in splenocyte proliferation post-pseudofracture trauma in comparison to unmanipulated controls (48102.9±4808.1 c.p.m.) after concanavalin A stimulation. FIG. 9B shows the splenocyte proliferative capacity after antiCD3e stimulation and a similar significant decrease (35245.2±6144.1 c.p.m.) post-pseudofracture trauma is seen in comparison to unmanipulated controls (54871.3±5271.8 c.p.m.)

This decrease in comparison with uninjured controls was also seen in the groups treated with laparotomy alone, consistent with the fact that the laparotomy procedure is itself a significant trauma.

As would be expected, the combined injury of PF+laparotomy also resulted in a significant decrease in splenocyte proliferative capacity in comparison with uninjured controls (FIG. 9A shows results after concanavalin A stimulation; FIG. 9B shows the data after antiCD3e stimulation).

In contrast, splenocytes from the injury groups that received NOX-100 proliferated at a rate similar to that of splenocytes from uninjured mice, indicating that the NOX-100 reversed the immune dysfunction induced by injury. FIG. 9A shows the increase (p=0.062) in proliferative capacity in the mice that underwent PF+laparotomy with NOX-100 (33658.6±3787.9 c.p.m.) above the proliferative capacity of those mice that underwent the same combined trauma and received vehicle (20461.9±5254.9 c.p.m.). FIG. 9B shows a significant increase (p<0.05) between these two groups after antiCD3e stimulation; the mice that received the nitric oxide scavenger NOX-100 had a reversal of splenocyte proliferation (45208.9±4890.5 c.p.m.) in comparison to those that received the vehicle (34514.9±9409.1 c.p.m.).

Splenocyte proliferation capacities in the injury groups that received the vehicle were found to be similar to the injury alone groups, and were significantly decreased from both uninjured controls and the combined trauma group (PF+laparotomy) that received NOX-100.

From these results, it may be concluded that delayed immune dysfunction seen in severe trauma failed to develop in animals that received the nitric oxide scavenger NOX-100, consistent with excess, extracellular nitric oxide contributing to the immune dysfunction seen in severe trauma. These findings are consistent with the use of NO as a therapeutic target for immunomodulation to limit injury-induced immune suppression.

### 8. EXAMPLE

### NOX-100 Inhibits and Prevents Post Injury Immunodepression as Demonstrated by Interferon Gamma Secretion

### 8.1 Materials and Methods

Additional measurements of splenocyte responses at the late 48 hr time point included assessment of splenocyte interferon-gamma release, an established parameter of immune dysfunction. Measurements were taken in response to two T cell mitogens: concanavalin A (2.5 pg/ml) and antiCD3e (1 μg/ml), and total splenocyte cultures were used. Splenocyte culture supernatants were collected and assessed by ELISA. Statistical significance (p<0.05) was assessed by Student's t-test and ANOVA.

### 8.2 Results

FIGS. 10A-B show splenocyte release of interferon gamma at 48 hrs post injury. A significant decrease in splenocyte release of interferon gamma is seen at 48 hrs post injury in comparison to unmanipulated controls and this is reversed with the administration of NOX-100.

FIG. 10A shows the levels of interferon-gamma released by splenocytes after concanavalin A stimulation; a significant (p<0.05) decrease (158.06±31.75 pg/mL) was seen following pseudofracture trauma in comparison to unmanipulated controls (621.98±50.81 pg/mL). FIG. 10B illustrates the splenocyte interferon-gamma release after antiCD3e stimulation and a similar significant decrease (325.57±69.13 pg/mL) post-pseudofracture trauma is seen in comparison to unmanipulated controls (561.27±61.52 pg/mL).

This decrease in comparison with uninjured controls was also seen in the laparotomy alone groups, as the laparotomy procedure is itself a significant trauma. As would be expected, the combined injury of PF+laparotomy also resulted in a significant decrease in splenocyte interferon-gamma release capacity in comparison with uninjured controls (FIG. 10A shows results after concanavalin A stimulation; FIG. 10B shows the data after antiCD3e stimulation).

In contrast, splenocytes from the injury groups that received NOX-100 proliferated at a rate similar to that of splenocytes from uninjured mice, indicating that the NOX-100 reversed the immune dysfunction induced by injury. FIG. 10A shows the significant increase (p<0.05) in capacity of splenocytes to release interferon-gamma in the mice that underwent PF+laparotomy with NOX-100 (636.28±85,04 pg/mL) above the capacity of those mice that underwent the same combined trauma and received vehicle (257.20±101.78 pg/mL). FIG. 10B shows a significant increase (p<0.05) between these two groups after antiCD3e stimulation; the mice that received the nitric oxide scavenger NOX-100 had a reversal of splenocyte cytokine release capacity (604.70±86.96 pg/mL) in comparison to those that received the vehicle (249.88±126.62 pg/mL).

Splenocyte interferon-gamma release capacities in the injury groups that received the vehicle were found to be similar to the injury alone groups, and were significantly decreased from both uninjured controls and the combined trauma group (PF+laparotomy) that received NOX-100.

In conclusion, these findings are consistent with the observations described above, and support a role of nitric oxide signaling in late post-traumatic immune dysfunction.

Various publications are cited herein, the contents of which are incorporated by reference in their entireties.

What is claimed is:

1. A method of inhibiting post-trauma immunodepression in a subject who has suffered a traumatic injury, comprising administering, to the subject, an effective amount of an agent that inhibits the effects of nitric oxide.

2. The method of claim 1, wherein the agent is a nitric oxide scavenger.

3. The method of claim 2, wherein the nitric oxide scavenger comprises a dithiocarbamate compound.

4. The method of claim 1, wherein the agent is NOX-100.

5. The method of claim 1, wherein the agent inhibits inducible nitric oxide synthase.

6. The method of claim 1, wherein the agent is N-(3-(aminomethyl)benzyl)acetamidine.

7. A method for inhibiting a decrease in cell-mediated immunity in a subject who has suffered a traumatic injury, comprising administering, to the subject, an effective amount of an agent that inhibits the effects of nitric oxide.

8. The method of claim 7, wherein the agent is a nitric oxide scavenger.

9. The method of claim 8, wherein the nitric oxide scavenger comprises a dithiocarbamate compound.

10. The method of claim 7, wherein the agent is NOX-100.

11. The method of claim 7, wherein the agent inhibits inducible nitric oxide synthase.

12. The method of claim 7 wherein the agent is N-(3-(aminomethyl)benzyl)acetamidine.

13. A method for reducing the risk of infection in a subject who has suffered a traumatic injury, comprising administering, to the subject, an effective amount of an agent that inhibits the effects of nitric oxide.

14. The method of claim 13, wherein the agent is a nitric oxide scavenger.

15. The method of claim 14, wherein the nitric oxide scavenger comprises a dithiocarbamate compound.

16. The method of claim 13, wherein the agent is NOX-100.

17. The method of claim 13, wherein the agent inhibits inducible nitric oxide synthase.

18. The method of claim 13 wherein the agent is N-(3-(aminomethyl)benzyl)acetamidine.

19. The method of claim 13 where the infection is selected from the group consisting of wound infection, pulmonary infection and sepsis.

20. A method of inhibiting post-trauma immunodepression in a subject who is expected to suffer a surgical traumatic injury, comprising administering, to the subject, an effective amount of an agent that inhibits the effects of nitric oxide.

21. The method of claim 20, wherein the agent is a nitric oxide scavenger.

22. The method of claim 21, wherein the nitric oxide scavenger comprises a dithiocarbamate compound.

23. The method of claim 20, wherein the agent is NOX-100.

24. The method of claim 20, wherein the agent inhibits inducible nitric oxide synthase.

25. The method of claim 20 wherein the agent is N-(3-(aminomethyl)benzyl)acetamidine.

26. A method of inhibiting post-sepsis immunodepression in a subject who has developed sepsis, comprising administering, to the subject, an effective amount of an agent that inhibits the effects of nitric oxide.

27. The method of claim 26, wherein the agent is a nitric oxide scavenger.

28. The method of claim 27, wherein the nitric oxide scavenger comprises a dithiocarbamate compound.

29. The method of claim 26, wherein the agent is NOX-100.

30. The method of claim 26, wherein the agent inhibits inducible nitric oxide synthase.

31. The method of claim 26 wherein the agent is N-(3-(aminomethyl)benzyl)acetamidine.
